(19) United States
(12) Patent Application Publication
(10) Pub. No.: US 2006/0222703 A1
(43) Pub. Date: Oct. 5, 2006

(54) Pharmaceutical composition and preparation method thereof

(75) Inventor: Giovanni Politi, Espoo (FI)

Correspondence Address: BANNER & WITCOFF, LTD., 28 STATE STREET, 28th FLOOR, BOSTON, MA, 02109-9601, US

(73) Assignee: IPRBOX Oy, Espoo (FI)

(21) Appl. No.: 11/097,969

(22) Filed: Apr. 1, 2005

(51) Int. Cl. A61K 31/198; B27N 3/00; A61K 9/20

(52) U.S. Cl. 424/464; 514/567; 264/109

(57) ABSTRACT

The invention relates to an oral solid pharmaceutical composition comprising pharmacologically effective amounts of entacapone, levodopa and carbidopa, or a pharmaceutically acceptable salt or hydrate thereof, and one or more pharmaceutically acceptable excipients, wherein the excipients are long-chain polymers having an equilibrium moisture content of at least 2%, and to a preparation method thereof. The compositions can be used for the treatment of Parkinson's disease.

### FIELD OF THE INVENTION

The present invention relates to a new pharmaceutical composition and to a new method for the preparation of such a composition. More particularly, the invention relates to a new oral solid pharmaceutical composition comprising entacpone, levodopa and carbidopa or a pharmaceutically acceptable salt or hydrate thereof. The pharmaceutical compositions of the invention are useful in the therapy of Parkinson's disease.

### BACKGROUND OF THE INVENTION

In the treatment of Parkinson's disease, the most commonly used dosage form is a mixture of levodopa and carbidopa, for example in the form of tablets. In the recent time, a new drug, namely entacapone, has been developed which improves the effect of levodopa and carbidopa. Entacapone is described in U.S. Pat. No. 5,446,194 as a catechol-O-methyltransferase (COMT) inhibitor. The publication also discloses that, for the treatment of Parkinson's disease, entacapone is given with levodopa, each in its own composition or combined in one composition. The publication further discloses enteral and parenteral routes of administration of entacapone. An oral composition containing entacapone and croscarmellose sodium is nowadays available under the trademarks COMTESS® and COMTAN® manufactured by Orion Corporation, Finland. Levodopa and carbidopa are the most commonly used drugs in the treatment of Parkinson's disease. Levodopa and carbidopa are commercially available as a combination tablet, e.g., under the trademarks NACOM®, ISICOM®, SINEMET® and SINEMET®LP 25, all distributed by DuPont Pharma, UK.

In the treatment regimen of parkinsonism, medicament needs to taken several times a day to keep the patients without symptoms. In the regimen where two separate tablets, i.e. one containing levodopa-carbidopa and the other containing entacapone, is problematic for many patients, such as those with tremor and old age. In other words, the regimen is in no way patient-friendly. The patient compliance has been improved by combining these three active agents, i.e. levodopa, carbidopa and entacapone, together. WO 01/01984 discloses an oral solid pharmaceutical composition comprising levodopa, carbidopa and entacapone and a pharmaceutically acceptable excipient, wherein carbidopa is separated from entacapone and levodopa. In that way, the bioavailability of carbidopa was reported to increase. It is further stated in the publication that bioavailability of carbidopa is significantly affected by a method of preparing of the composition. Both wet and dry granulations of the three active agents are suggested but the wet granulation is preferred. The publication teaches away from compaction granulation (dry granulation) by stating that when compaction granulation is used, large amounts of excipients are needed to obtain compressible granules and tablets having the desired, fast, dissolution behaviour of an immediate release formulation. Further, it is reported that when compaction granulation is used for preparing a fixed dose combination tablet containing levodopa, carbidopa and entacapone, the tablets became too large in size, especially for patients who have difficulties in swallowing. Moreover, the formulations prepared by compaction granulation of said publication are not acceptable because of poor stability.

It is further reported in WO 01/01984 that many commonly used excipients are not suitable for solid compositions containing entacapone, levodopa and carbidopa. Most of the levodopa-carbidopa formulations available in the market contain microcrystalline cellulose as a carrier. The authors of WO 01/01984 found, however, that microcrystalline cellulose destabilized the formulations on a long-term storage, where said three active agents were combined together.

The Applicant has now found that many disadvantages are associated with the wet granulation manufacturing disclosed in WO 01/01984. They are troublesome to perform and the necessity to use much excipients adds costs. Large amounts of excipients make the tablet formulations large in size which does not render the products patent-friendly due to swallowing problems, particularly for patients with tremor and old-age. Furthermore, the use of water when preparing a pharmaceutical composition by a wet granulation method causes several problems, relating to disintegration, dissolution, bioavailability and stability of the composition. During the wet granulation manufacturing, absorption-desorption fenomena occur which are generated when the composition comprises very dry agents, like entacapone and levodopa, and hygroscopic agents, like starch, closed together. These fenomena have a significant negative influence on the stability of the composition. Also, the additional water needed during the wet granulation of the composition, as described in the above publication, generates strong bonds between the different substances of the composition. Thus, in order to improve the disintegration of the tablets, there is need for use of strong disintegrants. Also, the dissolution of levodopa, carbidopa and entacapone is negatively influenced by the additional water. Furthermore, the Applicant has found that additional water gives rise to moisture balance problems in the pharmaceutical compositions described in WO 01/01984 thus deteriorating the stability of the compositions.

In the treatment of parkinsonism, it is highly desirable that the active agents, entacapone, levodopa and carbidopa or a pharmaceutically acceptable salt or hydrate thereof, are released from the oral composition as soon as possible after the intake. It is also highly desirable that the absorption of the three different active agents from one and the same oral solid composition corresponds to that of a commercial available entacapone tablet and that of a levodopa-carbidopa tablet in gastrointestinal tract. However, it is very challenging to adjust the absorption of three different active agents.

### BRIEF DESCRIPTION OF THE INVENTION

It is thus an object of the present invention to provide a new pharmaceutical oral solid composition and a new method in order to overcome the above problems so as to alleviate the above disadvantages. The objects of the invention are achieved by a composition and a method which are characterized by what is stated in the independent claims. The preferred embodiments of the invention are disclosed in the dependent claims.

Advantages of the pharmaceutical oral solid compositions of the present invention are their small size, fast disintegration and dissolution, and bioequivalency with the most common commercial compositions of levodopa-carbidopa and entacapone. Moreover, the compositions of the present invention are easy to coat and show good stability characteristics. Advantages of the method of the invention are good stability and velocity of the manufacturing and saving of costs due to the need of smaller amounts of source materials and to the simplicity of the production equipment involved.

The invention is based on the idea that by choosing appropriate pharmaceutical acceptable excipients for the pharmaceutical oral solid composition the above mentioned advantages can be achieved.

### DETAILED DESCRIPTION OF THE INVENTION

The Applicant has surprisingly found that a pharmaceutical oral solid composition comprising entacapone, levodopa and carbidopa or a pharmaceutically acceptable salt or hydrate thereof can be prepared without additional solvent, particularly water, said composition having bioequivalency at a level of already marketed products, small size and fast disintegration. These are important factors when considering the patient compliance on one hand and the absorption mechanism of said three drugs on the other hand. In addition, the compositions of the present invention are stable on a long-term storage due to the absent of additional water brought to the tablets by manufacturing method thereof.

In wet granulation methods, generally, water or solvents are used in order to improve the compressibility of a pharmaceutical formulation, like a tablet. The Applicant has found that the compressibility of all three active agents, i.e. entacapone, levodopa and carbidopa used in the present invention, is good. Thus, no water or any other solvent is needed for altering physical properties of said active agents for compressiblity. No auxiliary compression aids are necessary either. According to the present invention, by means of selection of suitable excipients an oral solid pharmaceutical composition comprising entacapone, levodopa and carbidopa can be prepared by a solvent-free method.

Thus, the present invention provides an oral solid pharmaceutical composition comprising pharmacologically effective amounts of entacapone, levodopa and carbidopa, or a pharmaceutically acceptable salt or hydrates thereof, and one or more pharmaceutically acceptable excipients, wherein the excipients are long-chain polymers having an equilibrium moisture content of at least 2%. The long-chain polymeric compounds useful in the composition of the present invention include, e.g., microcrystalline cellulose and starch. Starch is preferably maize starch. In a preferred embodiment of the invention, maize starch is used as an excipient. Further, in a preferred embodiment of the invention, the equilibrium moisture content of the excipients is at least 3.5%. The equilibrium moisture content of microcrystalline cellulose and maize starch is in the range of 4 to 5% and 4 to 9%, respectively. Hydrogen bonds necessary to hold the composition of the invention together are mainly formed by the naturally existing water brought to the composition by the excipient(s) used. The equilibrium moisture content of the excipients used in the present invention is measured by a weightloss method described in European Pharmacopoeia, 4^{th} edition 2002, page 48.

The total amount of the excipients in the composition of the invention is at most 40%, preferably 10 to 35%, based on the dry weight of the composition.

The total moisture content of the composition of the invention must be at least 1 wt %, preferably 2 to 3 wt %. The total moisture content T is a theoretical value calculated according to an equation given below:

$$\frac{\begin{matrix}0.5 \times L + 0.5 \times C + 0.5 \times E + ( H ) \times M + \\ ( H 1 ) \times {S}_{1} + ( H 2 ) \times {S}_{2} + … + ( Hn ) \times {S}_{n}\end{matrix}}{L + C + E + M + {S}_{1} + … + {S}_{n}} = T$$

Where L=an amount of levodopa in mg

C=an amount of carpidopa in mg

E=an amount of entacapone in mg

M=an amount of a lubricant in mg

S₁ to Sₙ=amounts of excipients in mg

H=an equilibrium moisture content of a lubricant

H1 to Hn=an equilibrium moisture content of the excipients

In the above equation, it is assumed that the moisture content of levodopa, carbidopa and entacapone is 0.5%.

The pharmacologically effective amounts of entacapone, levodopa and carbidopa, or a pharmaceutically acceptable salt or hydrate thereof, in the composition of the present invention can all be in the range of those already existing in the market worldwide. The amounts of said active agents depend on numerous factors known to one skilled in the art, such as the severity of the condition of the patient, the desired duration of use, etc. The amount of entacapone in the composition of the invention can vary in the range of 25 to 400 mg, preferably 25 to 300 mg, more preferably 50 to 200 mg, most preferably 200 mg. The amount of levodopa can vary in the range of 25 to 300 mg, more preferably 50 to 250 mg. The amount of carbidopa can vary in the range of 5 to 75 mg, more preferably 10 to 50 mg. An oral solid composition of the present invention can thus contain, e.g., 200 mg of entacapone, 100 mg of levodopa and 25 mg of carbidopa, or 200 mg of entacapone, 150 mg of levodopa and 37.5 mg of carbidopa.

The oral solid compositions of the present invention can contain a suitable conventional binder and/or lubricant in amounts known in the art. Examples of suitable lubricants useful in the present invention include, e.g. magnesium stearate, calcium stearate, hydrogenated vegetable oil, talc. etc. Preferably magnesium stearate is used. The amount of the lubricant can vary in the range of 0.5% to 1.5%.

Due to the optimal consistency of the composition of the present invention, achieved by a selection of appropriate excipients, no disintegrants need to be added to the final product having desirable disintegration behaviour. This is an obvious advantage resulting in saving source materials and enabling to make smaller tablets.

An oral solid pharmaceutical composition of the present invention can exist in variety of shapes, like in tablets and capsules. Preferably, the composition of the invention is in the form of tablets, preferably in oval form.

In another aspect of the invention, a solvent-free method method of preparing an oral solid pharmaceutical composition comprising pharmacologically effective amounts of entacapone, levodopa and carbidopa, or a pharmaceutically acceptable salt or hydrate thereof, and one or more pharmaceutically acceptable excipients is provided, which comprises

a) simultaneous mixing of pharmaceutically effective amounts of entacapone, levodopa and carbidopa, or a pharmaceutically acceptable salt or hydrate thereof, together with at least one pharmaceutically acceptable excipient which are ong-chain polymers having an equilibrium moisture content of at least 2% to obtain a first mixture,

b) granulating the first mixture to obtain granules, if necessary;

c) adding a lubricant to the first mixture of step a) or to the granules of step b) to obtain a second mixture;

d) formulating the second mixture into an oral solid composition; and

e) if desired, coating the composition obtained in step d).

The method of the invention is preferably a compaction granulation.

The oral solid compositions of the invention are preferably coated in order to improve the stability of the composition and to avoid undesirable discoloration in the mouth. Coating is easy to carry out since no disintegrants are needed in the composition of the invention. Coating can be performed by using as coating agents those conventional in the art, like HPMC-coating.

The mixing step before granulation can be conducted, e.g. in a high shear mixter of in a fluiduzed bed, but preferably in a tumbler mixer in a manner known in the art.

According to the method of the present invention, it is possible to prepare formulations which are substantially smaller in size than the prior art formulations. The weight of the formulation of the invention usually varies in the range of about 350 mg to 530 mg.

Dissolution and disintegration Tests in vitro

Disintegration of the formulations of the present invention was tested by a method described in European Pharmacopoeia, 4^{th} edition 2002, page 191 by means of a standard Erweka dissolution tester. The formulations 1 to 4 of the present invention are described below. For comparison, a tablet prepared according to WO 01/01984 and SINEMET® and COMTAN® tablets were also tested.

| | | WO 01/01984 | | |
|---|---|---|---|---|
| | Invention | wet granulation | SINEMET ® | COMTAN ® |
| disintegration | ca. 1 min | ca. 8 min | ca. 1 min | ca. 1 min |
| time | | | | |
| dissolution time: | | | | |
| levodopa and | ca. 100% in 10 | ca. 95% in 45 | ca. 100% in 10 | |
| cabidopa | minutes | minutes | minutes | |
| entacapone | ca. 100% in 20 | ca. 90% in 45 | | ca. 96% in 30 |
| | minutes | minutes | | minutes |

Times given for dissolution of the formulations of the present invention are estimated values. Estimation is made taking into account various ingredients included in the compositions, manufacturing method and disintegration times of the composition. Estimation can be regarded as justified, since disintegration time of the formulations of the invention corresponds to that of SINEMET® and COMTAN® tablets and the particle size of the active agents are equal in the compositions of the invention and in SINEMET® and COMTAN® tablets.

It can be seen that the dissolution and disintegration of the tablets of the present invention is significantly better that those of the prior art formulation.

Bioequivalency

Plasma concentrations of entacapone from COMTESS®, and levodopa and carbidopa from SINEMET® PLUS 100/25 and those of the compositions described in WO 01/01984 have been illustrated in said publication. COMTESS® and SINEMET® PLUS 100/25 formulations comprise considerable amounts of microcrystalline cellulose. Thus, it can justly presume that the bioequivalency of those compositions of the present invention comprising microcrystalline cellulose corresponds to that of said commercial formulations. Also, in case where maize starch is included in the compositions of the present invention, together or without cellulose, it can be assumed that the bioequivalency of the compositions of the intenvion are comparable to that of commercial formulations, based on the results obtained in WO 01/01984. This conclusion can be drawn since the disintegration of the compositions of the present invention, SINEMET® and COMTESS® in water, measured according to a method mentioned above, takes place at the same rate.

The invention will be further illustrated by the following non-limiting examples.

### EXAMPLES

The compositions containing entacapone, levodopa and carbidopa were formulated into tablets as follows: Entacapone, levodopa and carbidopa were mixed together with one or more excipients used, compacted and granulated. A lubricant is then added to the granules obtained. After the final mixing, the bulk was pressed into tablets.

With the procedure as described above, the following tablet formulations were prepared:

Formulation 1

| | | |
|---|---|---|
| Levodopa | 100.00 | mg |
| Carbidopa (monohydr. respond. | 27.00 | mg |
| Carbidopa 25.0 mg) | | |
| Entacapone | 200.00 | mg |
| Maize starch | 75.40 | mg |
| Microcrystalline cellulose, NF | 65.59 | mg |
| Magnesiumstearat | 5.00 | mg |

The above formulation has the following characteristics: weight: 473 mg, moisture content: ca. 2.2%, hardness: 5.2-7.8 kp, compactability: good, tabletability: good, flowability: good.

Formulation 2

| | | |
|---|---|---|
| Levodopa | 100.00 | mg |
| Carbidopa (monohydr. respond. | 27.00 | mg |
| Carbidopa 25.0 mg) | | |
| Entacapone | 200.00 | mg |
| Maize starch | 135.40 | mg |
| Microcrystalline cellulose, NF | 65.59 | mg |
| Magnesiumstearat | 5.00 | mg |

The above formulation has the following characteristics: weight: 533 mg, moisture content: ca. 3.1%, hardness: 8.9-11.4 kp, compactability: good, tabletability: good, flowability: good.

Formulation 3

| | | |
|---|---|---|
| Levodopa | 100.00 | mg |
| Carbidopa (monohydr. respond. | 27.00 | mg |
| Carbidopa 25.0 mg) | | |
| Entacapone | 200.00 | mg |
| Maize starch | 100.00 | mg |
| Magnesiumstearat | 4.00 | mg |

The above formulation has the following characteristics: weight: 431 mg, moisture content: ca. 2.0%, hardness: 4.5-6.2 kp, compactability: good, tabletability: good, flowability: good.

Formulation 4

| | | |
|---|---|---|
| Levodopa | 100.00 | mg |
| Carbidopa (monohydr. respond. | 27.00 | mg |
| Carbidopa 25.0 mg) | | |
| Entacapone | 200.00 | mg |
| Maize starch | 90.00 | mg |
| Microcrystalline cellulose, NF | 30.00 | mg |
| Magnesiumstearat | 5.00 | mg |

The above formulation has the following characteristics: weight: 452 mg, moisture content: ca. 2.3%, hardness: 5.67.9 kp, compactability: good, tabletability: good, flowability: good.

It will be obvious to a person skilled in the art that, as the technology advances, the inventive concept can be implemented in various ways. The invention and its embodiments are not limited to the examples described above but may vary within the scope of the claims.

1. An oral solid pharmaceutical composition comprising pharmacologically effective amounts of entacapone, levodopa and carbidopa, or a pharmaceutically acceptable salt or hydrate thereof, and one or more pharmaceutically acceptable excipients, wherein the excipients are long-chain polymers having an equilibrium moisture content of at least 2%.

2. A composition of claim 1, wherein the equilibrium moisture content of the excipients is at least 3.5%

3. A composition of claim 1, wherein the excipients used are maize starch and microcrystalline cellulose, preferably maize starch.

4. A composition of claim 1, wherein the total amount of the excipients is at most about 40%, preferably 10 to 35%, based on the dry weight of the composition.

5. A composition of claim 4, wherein the amount of maize starch is about 23% based on the dry weight of the composition.

6. A composition of claim 1, wherein the total moisture content of the composition is at least 1 wt %, preferably 2 to 3 wt %.

7. A composition of claim 1, wherein the composition does not contain any disintegrants.

8. A solvent-free method of preparing an oral solid pharmaceutical composition comprising pharmacologically effective amounts of entacapone, levodopa and carbidopa, or a pharmaceutically acceptable salt or hydrate thereof, and one or more pharmaceutically acceptable excipients, which comprises
a) simultaneous mixing of pharmaceutically effective amounts of entacapone, levodopa and carbidopa, or a pharmaceutically acceptable salt or hydrate thereof, together with at least one pharmaceutically acceptable excipient having an equilibrium moisture content of at least 2% to obtain a first mixture,
b) granulating the first mixture to obtain granules, if necessary;
c) adding a lubricant to the first mixture of step a) or to the granules of step b) to obtain a second mixture;
d) formulating the second mixture into an oral solid composition; and
e) if desired, coating the composition obtained in step d).

9. A method of claim 8, wherein the method is compaction granulation.

10. A method of treating Parkinson's disease which comprises administering to a host in need of the treatment a composition according to claim 1.
